(19) United States
(12) Patent Application Publication
(10) Pub. No.: US 2022/0332902 A1
(43) Pub. Date: Oct. 20, 2022

(54) RADICAL CROSSLINKED ZWITTERIONIC GELS AND USES THEREOF

(71) Applicants: Colorado School of Mines, Golden, CO (US); GelSana Therapeutics, Inc., Aurora, CO (US)

(72) Inventors: Melissa D. Krebs, Aurora, CO (US); Michael A. Stager, Golden, CO (US); Matthew Osmond, Golden, CO (US); Matthew Davidson, Aurora, CO (US)

(21) Appl. No.: 17/716,538

(22) Filed: Apr. 8, 2022

Related U.S. Application Data
(60) Provisional application No. 63/173,017, filed on Apr. 9, 2021.

(51) Int. Cl. C08J 3/075; C08F 220/28; C08F 220/38; A61L 26/00

(52) U.S. Cl. CPC C08J 3/075; C08F 220/281; C08F 220/387; A61L 26/0004; A61L 26/0066; A61L 26/008; A61L 26/0014; A61L 2400/06; A61L 2300/602; A61L 2300/412; C08J 2333/14; C08J 2433/14

(57) ABSTRACT

The present invention provides radical crosslinked zwitterionic gels, methods of preparing the radical crosslinked zwitterionic gels, and methods of using the radical crosslinked zwitterionic gels for treating a wound.

### CROSS REFERENCE TO RELATED APPLICATIONS

This application claims the benefit of U.S. provisional patent application No. 63/173,017, which was filed on Apr. 9, 2021, and is incorporated herein in its entirety.

### FIELD OF THE INVENTION

The present disclosure generally relates to radical crosslinked zwitterionic gels, methods for preparing the radical crosslinked zwitterionic gels, compositions comprising the radical crosslinked zwitterionic gels, and methods of treating a wound using the radical crosslinked zwitterionic gels.

### BACKGROUND OF THE INVENTION

The skin is the largest organ of the human body, and protects the body from microbial invasion and other exterior damages. This organ, however, can be damaged (i.e. wounds), which are generated by mechanical or thermal damage. Wounds can be life threatening, depending on the size and depth of the wound. When wounds are difficult to heal, they can become chronic due to dysregulation of inflammation.

Chronic wounds can be caused by, among other things, diabetes, burns, immunological states and vascular insufficiency. In diabetic patients, for example, healing impairment is caused by neuropathy, ischemia, and/or trauma. These factors can lead to opportunities for infections to populate the area, which may cause life-threatening infections. Chronic skin wounds are a critical problem that is reaching epidemic proportions; they are estimated to affect 20-60 million people worldwide by 2026. Unlike acute wounds, which heal after a certain period of time, chronic skin wounds heal slowly (in 8 weeks or more) or not at all. Chronic wounds can lead to long-term hospitalization, which entails a high burden on the health care system due to medical costs associated with wound care products, surgery, and physician and nursing resources. Medical assistance does not prevent serious complications such as foot amputation, morbidity, and mortality, as no efficient therapies have been developed. In fact, the 5-year mortality rate of chronic skin wounds is comparable to or worse than that of some common types of cancer, including prostate, breast, and colon cancers.

Hydrogels have long been considered as promising materials for wound dressing materials due to good oxygen permeation and a high water content that can help maintain a moist environment around the wound. More importantly, therapeutic molecules such as growth factors or antibiotics can be readily loaded into the hydrogels to promote wound healing and to protect the wound from bacterial infections and promote the healing process. Unfortunately, these hydrogels generally result in a rapid release of therapeutic molecules (typically a few hours) with a large release due to a highly porous structure of the gels. A rapid release of therapeutics not only decreases the efficiency of the therapy, but can also cause side effects due to the sudden increase in the blood concentrations of these molecules.

What is needed is a hydrogel that is conveniently prepared, has enhanced properties such as high water content, good oxygen permeation, low degradation rate, bioinertness, and cytocompatability, and will allow for a controlled release of a therapeutic agent over an extended period.

### SUMMARY OF THE INVENTION

The invention generally relates to dressings that are useful for administering active ingredients to the wound. More particularly, it relates to crosslinked zwitterionic gel dressings having water-soluble wound facing layers comprising a zwitterionic monomer and a non-zwitterionic monomer; methods of using such dressings for the controlled or sustained delivery of active ingredients to wounds; and methods for their manufacture. The wound dressings of the invention can provide for the controlled and sustained release of an active ingredient for at least about 12 hours.

In one aspect, disclosed herein, are radical crosslinked zwitterionic gels, the radical crosslinked zwitterionic gel comprising a zwitterionic monomer selected from the group consisting of [2-(methacryloyloxy) ethyl]dimethyl-(3-sulfopropyl)ammonium hydroxide (SBMA), [(methacryloyloxy)ethyl] dimethylammonio]propionate (CBMA), 2-methacryloyloxyethyl phosphorylcholine (MPC), and combinations thereof; and a non-zwitterionic monomer. In some embodiments, the zwitterionic monomer and the non-zwitterionic monomer are at least partially crosslinked. In some embodiments, the radical crosslinked zwitterionic gel has an average pore size greater than about 50 mm and less than about 100 mm.

In various embodiments, the radical crosslinked zwitterionic gels can be formed as a hydrated gel, a lyophilized powder that can be hydrated as needed, or a lyophilized foam.

In some embodiments, crosslinked zwitterionic gels of the invention are characterized by highly tunable mechanical properties. That is, the invention provides technologies that permit production and use of crosslinked zwitterionic gels are characterized by mechanical properties (e.g., pore size, strength [e.g., as assessed by storage modulus], flexibility, stiffness, etc.) that are particularly suitable for use in supporting cell growth, function, viability, and/or differentiation. For example, in some embodiments, the crosslinked zwitterionic gels are characterized by mechanical properties that support living cells, including, for example as evidenced by outgrowth of extensions on human mesenchymal stem cells.

In some embodiments, the crosslinked zwitterionic gels of the invention are characterized by a desired high storage modulus value. In some embodiments, a high storage modulus value corresponds with a strong and/or robust hydrogel. In some embodiments, the crosslinked zwitterionic gels include a high storage modulus value. In some particular embodiments exemplified herein, provided covalently crosslinked hydrogels are characterized by a storage modulus value between about 50 Pa and about 5,000 Pa without showing an indication of a plastic deformation. In some particular embodiments exemplified herein, the crosslinked zwitterionic gels are characterized by a storage modulus value great than about 1 kPa without showing an indication of a plastic deformation. In some embodiments, crosslinked zwitterionic gels of the invention are characterized in that they recover from a shear strain of at least 100% while resisting degradation and without showing evidence of a plastic deformation.

In some embodiments, the crosslinked zwitterionic gels are characterized by high elasticity. Elasticity or elastic deformation generally measures a tendency of a material to return to its original size and/or shape after a force having been applied to the material and having deformed the material is subsequently removed. In contrast, plastic deformation follows application of enough stress on a material to cause a change in the size and/or shape of the material in a way that is not reversible, such that the material does not return to its original size and/or shape. A plastic deformation specifically involves a change to the structure of the material, such as a molecular and/or atomic shift or dislocation from which the material cannot recover. In some embodiments, the crosslinked zwitterionic gels of the present invention are characterized as having a tangent modulus value between about 50 Pa to about 5,0000 Pa without showing an indication of a plastic deformation. In some embodiments, covalently crosslinked, hydrogels of the present invention are characterized in that they recover from a compressive strain of at least 75% while resisting degradation and without showing evidence of a plastic deformation.

In some embodiments, the crosslinked zwitterionic gels are characterized by a high resiliency. Resiliency provides an indication of an ability of a material to absorb energy when a force is applied and the material is deformed and subsequently release energy when the force is removed permitting the material to return to its natural state. In some embodiments, the crosslinked zwitterionic gels of the invention are characterized as highly resilient to a repetitive force with high cycle. In some embodiments, the crosslinked zwitterionic gels of the invention are characterized in that they recover from exposure to a compressive strain of at least 10% without showing evidence of a deformation. In some embodiments, the crosslinked zwitterionic gels of the invention are characterized in that they recover from exposure to a shear strain of at least 10% without showing evidence of a plastic deformation.

In some embodiments, the crosslinked zwitterionic gels are configured to support encapsulation of at least one therapeutic active agent. In some embodiments, the crosslinked zwitterionic gels may encapsulate or otherwise comprise at least one therapeutic active agent. In some embodiments, the crosslinked zwitterionic gels that comprise at least one therapeutic active agent may release the agent in a slow or sustained release manner for a period of at least or about 6 hours, at least or about 12, hours, at least or about 18 hours, at least or about 24 hours, at least or about 48 hours, 72 hours or at least or about 96 hours.

In some embodiments, the crosslinked zwitterionic gels are configured to support incorporation of and/or modification with one or more functional moieties. In some embodiments, the crosslinked zwitterionic gels of the invention provide tunable mechanical properties that support, for example for cell engineering and/or tissue regeneration applications including for example in the treatment or prevention of a disease, disorder or condition and/or for inducing tissue repair.

In another aspect, provided herein, are methods to prepare the radical crosslinked zwitterionic gel.

In yet another aspect, provided herein, are methods of treating a wound. The methods comprise administering or applying the radical crosslinked zwitterionic gel to a wound in a subject in need thereof.

Other features and iterations of the invention are described in more detail below.

### BRIEF DESCRIPTION OF THE DRAWINGS

FIGS. 1a and 1b illustrates photographs of the radical crosslinked zwitterionic gels prepared in white light and at 405 nm light.

FIG. 2 is a graph of the percentage of cell viability versus various radical crosslinked zwitterionic gels.

FIG. 3 is a series of photographs comparing the antifouling of the radical crosslinked zwitterionic gels compared to a glass slide.

FIG. 4a is a graph illustrating the storage modulus of various radical crosslinked zwitterionic gels versus ambient and UV light.

FIG. 4b is a graph illustrating the shear storage modulus of various radical crosslinked zwitterionic gels.

FIG. 4c is a graph illustrating the shear storage modulus of various 25% and 50% radical crosslinked zwitterionic gels.

FIG. 4d is a graph illustrating the shear storage modulus of various 25% and 50% radical crosslinked zwitterionic gels.

FIG. 5 is a graph illustrating the swelling ratio of radical crosslinked zwitterionic gels over 14 days.

FIG. 6 is a graph illustrating the degradation ratio of radical crosslinked zwitterionic gels over 14 days.

FIG. 7 shows a series of photographs on the injectability, stretchability, compression and release, and self-healing characteristics of the radical crosslinked zwitterionic gels prepared in white light.

FIG. 8 shows a series of photographs on the injectability, stretchability, compression and release, and self-healing characteristics of the radical crosslinked zwitterionic gels prepared in 405 nm light.

FIG. 9 is a series of photographs comparing the injectability of the radical crosslinked versus the cryo zwitterionic gels.

FIG. 10a are graphs that show the release of myoglobin of various radical crosslinked zwitterionic gels over an extended period of time.

FIG. 10b are graphs that show release of cerium oxide nanoparticles (CNP) from various radical crosslinked zwitterionic gels over an extended period of time.

FIG. 11 is a series of photographs illustrating the wound closure of various radical crosslinked zwitterionic gels over a 16-day period.

FIGS. 12a and 12b are graphs illustrating the percentage wound closure and full wound closure in mice.

FIG. 13 illustrates photographs of zwitterionic gels containing methacrylated hyaluronic acid.

FIG. 14 illustrates photographs of zwitterionic gels fabricated with SBMA, HEMA, LAP, and/or PEGDMA.

FIG. 15 illustrates photographs of zwitterionic hydrogels fabricated without and with resveratrol (an antioxidant).

FIG. 16 illustrates photographs of zwitterionic hydrogels fabricated with ammonium persulfate and TEMED as the radical initiators.

FIG. 17 illustrates photographs of photocrosslinked hydrogels with varying amounts of ascorbic acid added during polymerization.

FIG. 18 illustrates quantitative tissue adhesive strength data for various zwitterionic hydrogels.

FIG. 19 illustrates photographs of lyophilized and rehydrated zwitterionic hydrogel samples.

### DETAILED DESCRIPTION OF THE INVENTION

In one aspect, the present disclosure provides radical crosslinked zwitterionic gels. These gels are easily prepared, have many desirable attributes such as highly stretchable, injectability, low degradation over time, bio-inertness, and highly sustained released of therapeutic agents. In certain embodiments, the gels of the disclosure may prevent protein absorption during use.

### Definitions

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

As used herein, the term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages.

The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

The term “controlled release” as used herein refers to a drug-containing formulation in which the manner and profile of drug release from the formulation are controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that may result in substantially constant levels of a drug over an extended time period. The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.” The term “long-term” release, as used herein, means that the drug formulation is constructed and arranged to deliver therapeutic levels of the active ingredient for at least: 2 hours, 3 hours, 4 hours, hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, 73 hours, 74 hours, 75 hours, 76 hours, 77 hours, 78 hours, 79 hours, 80 hours, 81 hours, 82 hours, 83 hours, 84 hours, 85 hours, 86 hours, 87 hours, 88 hours, 89 hours, 90 hours, 91 hours, 92 hours, 93 hours, 94 hours, 95 hours, 96 hours, 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, or 120 hours.

The term “encapsulated” is used herein to refer to substances that are completely surrounded by another material.

As used herein, a “functional” biological molecule is a biological molecule in a form in which it exhibits a property and/or activity by which it is characterized. A biological molecule may have two functions (i.e., bi-functional) or many functions (i.e., multifunctional).

As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

As used herein, the term “hydrophilic” and/or “polar” refers to a tendency to mix with, or dissolve easily in, water.

As used herein, the term “hydrophobic” and/or “non-polar”, refers to a tendency to repel, not combine with, or an inability to dissolve easily in, water.

As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

The term “porosity” as used herein, refers to a measure of void spaces in a material and is a fraction of volume of voids over the total volume, as a percentage between 0 and 100%. A determination of a porosity is known to a skilled artisan using standardized techniques, for example mercury porosimetry and gas adsorption (e.g., nitrogen adsorption).

As used herein, the term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

The term “stable,” when applied to compositions herein, means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

The term “zwitterionic” is a neutral molecule with a positive and a negative electrical charge.

(I) Radical Crosslinked Zwitterionic Gels

The present disclosure encompasses radical crosslinked zwitterionic gels comprising a zwitterionic monomer and a non-zwitterionic monomer. In some embodiments that zwitterionic monomer is at least partially crosslinked with the non-zwitterionic monomer. In various embodiments, the crosslinked zwitterionic gels are easy to apply, are extrudable, stretchable and easily conform to wounds.

In some embodiments, the crosslinked zwitterionic gels of the invention are manufactured from commonly available reagents. In some embodiments, the crosslinked zwitterionic gels of the invention can be produced without needing volatile chemicals to induce gelation. In some embodiments, the crosslinked zwitterionic gels of the invention can be produced using all aqueous processing. In some embodiments, the crosslinked zwitterionic gels of the invention can be produced without the need to rely on expensive or complicated equipment (power supply, sonicator, vortexer, etc.). In some embodiments, the crosslinked zwitterionic gels of the invention are inexpensive to prepare, easy to prepare, and capable of bulk manufacturing.

In some embodiments, the crosslinked zwitterionic gels of the invention are prepared under mild, physiologically safe reaction conditions. In some embodiments, mild aqueous processing is amenable to incorporation of a therapeutic active agent during formation. In some embodiments, hydrogels provide for ease of infiltration of for example a therapeutic active agent. In some embodiments, the crosslinked zwitterionic gels of the invention are biocompatible and biodegradable. In some embodiments, the crosslinked zwitterionic gels are not cytotoxic. In some embodiments, the crosslinked zwitterionic gels are non-immunogenic.

In some embodiments, the crosslinked zwitterionic gels are characterized by superior resilience and elasticity. In some embodiments, the crosslinked zwitterionic gels of the invention are characterized in that they fully recover from large strains or long term cyclic compressions. In some embodiments, the crosslinked zwitterionic gels of the invention are characterized in that they withstand long term stress with negligible changes in modulus and without showing an indication of appreciable changes in mechanical properties, such as a plastic deformation. In some embodiments, the crosslinked zwitterionic gels of the invention are characterized in that they are capable of withstanding repeated strains. In some embodiments, the crosslinked zwitterionic gels of the invention that have been shown to exhibit the above identified characteristics and/or properties are formed from solutions of a zwitterionic monomer and a non-zwitterionic monomer.

In some embodiments, the crosslinked zwitterionic gels of the invention swell up to 50%, up to 100%, up to 150%, up to 200%, up to 300%, or up to 400% when exposed to solvents. In some embodiments, the crosslinked zwitterionic gels of the invention are configured to support693 therapeutic agent encapsulation. In some embodiments, the crosslinked zwitterionic gels of the invention provide direct encapsulation of one or more therapeutic agents. In some embodiments, the crosslinked zwitterionic gels show long term storage capability with or without a therapeutic agent.

The zwitterionic monomer may be [2-(methacryloyloxy) ethyl]dimethyl-(3-sulfopropyl)ammonium hydroxide (SBMA), [(methacryloyloxy)ethyl]dimethylammonio]-propionate (CBMA), 2-methacryloyloxyethyl phosphorylcholine (MPC), and combinations thereof. In one embodiment, the zwitterionic monomer is 2-(methacryloyloxy) ethyl] dimethyl-(3-sulfopropyl) ammonium hydroxide (SBMA).

The non-zwitterionic monomer may be hydroxyethyl methacrylate (HEMA).

In general, the weight ratio of the zwitterionic monomer to the non-zwitterionic monomer may range from about 1:19 to about 19:1. In various embodiments, weight ratio of the zwitterionic monomer to the non-zwitterionic monomer may range from 1:19 to about 19:1, from about 1:18 to about 18:1; 1:17 to about 17:1, from about 1:16 to about 16:1; 1:15 to about 15:1, from about 1:14 to about 14:1; 1:13 to about 13:1, from about 1:12 to about 12:1; 1:11 to about 11:1, from about 1:10 to about 10:1; 1:9 to about 9:1, from about 1:8 to about 8:1; 1:7 to about 7:1, from about 1:6 to about 6:1; 1:5 to about 5:1, from about 1:4 to about 4:1; 1:3 to about 3:1, from about 1:2 to about 2:1.

The radical crosslinked zwitterionic gels may further comprises an optional crosslinking agent. This crosslinking agent, after polymerization, provides additional crosslinking to the zwitterionic monomer and non-zwitterionic monomer. The crosslinking agent is selected from a group consisting of glycerol dimethacrylate (GDMA), methylene bisacrylamide (MBA), polyethylene glycol dimethacrylate (PEGDMA), and a combination of two or more crosslinkers. In one preferred embodiment, the crosslinking agent is polyethylene glycol dimethacrylate (PEGDMA).

Generally, the concentration percentage ratio of the crosslinking agent to the radical crosslinked zwitterionic gel may range from about 0.0:1.0 to about 0.25:1.0. In various embodiments, the concentration percentage ratio of the crosslinking agent to the radical crosslinked zwitterionic gel may range from about 0.0:1.0 to about 0.25:1.0, from about 0.001:1.0 to about 0.2:1.0, from about 0.005:1.0 to about 0.15:1.0, from about 0.01:1.0 to about 0.1:1.0.

The radical crosslinked zwitterionic gel may further comprise a rheology modifier. The rheology modifier further enhances the rheological properties of the radical crosslinked zwitterionic gel. Non-limiting examples of suitable rheology modifiers may be various clays. In one embodiment, the rheology modifier is synthetic smectic clay. In some embodiments, the crosslinked zwitterionic gel comprise additives, for example, therapeutic, preventative, and/or diagnostic agents.

The radical crosslinked zwitterionic gel may further comprise an antioxidant. The antioxidant further enhances the storage capabilities and enhances the properties of the radical crosslinked zwitterionic gel. Non-limiting examples of suitable antioxidants include resveratrol, various vitamins, uric acid, and glutathione. In one embodiment, the antioxidant is resveratrol. In other embodiments, the antioxidant may be A5G81 peptide; ascorbic acid (vitamin C); astaxanthin; capric acid; Cerium oxide nanoparticles; curcumin; cyaniding; epigallocatechin gallate; forskolin; gingerol; ginkgo biloba; glutamine; glutathione; glycyrrhizin; phosphatidylserine; piperine; quercetin; resveratrol; sulforaphane; superoxide dismutase; taurine; ubiquinol; uric acid; ursolic acid; vitamin E, etc. In other embodiments, the antioxidant may be an NDPH-oxidase inhibitor (e.g., 2-Acetylphenothiazine (ML171); apocynin; APX-115; G6PDi-1; GKT136901; GLX351322; GSK2795039; Setanaxib (GKT137831); VAS2870, etc.).

In some embodiments, the crosslinked zwitterionic gel comprise additives, for example, therapeutic, preventative, and/or diagnostic agents.

The radical crosslinked zwitterionic gel may further comprise a therapeutic agent. The therapeutic agent may be covalently bound through a hydroxyl group, ionically bound to one or more ions in the radical crosslinked zwitterionic gel, hydrogen bonded, bound through van der Walls interactions with the radical crosslinked zwitterionic gel, or a combination of two or more of these. The therapeutic agent, after administration or application, may be released over an extended period from the radical crosslinked zwitterionic gel, e.g., to enhance wound healing.

Any suitable therapeutic agent useful in the treatment of wounds may be used in connection with the radical crosslinked zwitterionic gels of the disclosure. Non-limiting therapeutic agents include myoglobin, cerium oxide nanoparticles (CNPs), miRNA, siRNA, mRNA, growth factors, and combinations thereof. By way of example, therapeutic agents may include silver, including colloidal silver; DNAse 1 (e.g., Dornase alfa); miRNA, siRNA, mRNA, (e.g., miRNA-146a); Citrullination inhibitors (e.g., C1-amidine); Growth Factors (e.g., VEGF, PDGF (e.g., Regranex), EGF, FGF, TGF-b, GM-CSF, and isoforms thereof); Hyaluronic acid; Tacrolimus and AMD3100; Insulin; Collagenase-Debridement; Broad spectrum antibiotics (e.g., gentamicin, vancomycin, trimethoprim/sulfamethoxazole (e.g. Bactrim), Amoxicillin/clavulanic acid (Augmentin), Ampicillin, Doxycycline, Minocycline, Aminoglycosides, Azithromycin, Carbapenems, Piperacillin/tazobactam, Quinolones (e.g. ciprofloxacin), Tetracycline-class drugs, Chloramphenicol, Ticarcillin), etc.

Other suitable therapeutic agents that may be used in connection with the radical crosslinked zwitterionic gels of the disclosure include an acid/alkaline-labile drug, a pH dependent drug, or a drug that is a weak acid or a weak base. Examples of acid-labile drugs include statins (e.g., pravastatin, fluvastatin and atorvastatin), antibiotics (e.g., penicillin G, ampicillin, streptomycin, erythromycin, clarithromycin and azithromycin), nucleoside analogs (e.g., dideoxyinosine (ddI or didanosine), dideoxyadenosine (ddA), dideoxycytosine (ddC)), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine.

In another embodiment, the therapeutic agent that may be used in connection with the radical crosslinked zwitterionic gels of the disclosure may be an antibacterial agent. Suitable antibacterial agents include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline).

In an alternate embodiment, the therapeutic agent that may be used in connection with the radical crosslinked zwitterionic gels of the disclosure may be an antiviral protease inhibitor (e.g., amprenavir, fosamprenavir, indinavir, lopinavir/ritonavir, ritonavir, saquinavir, and nelfinavir).

In some embodiments, an additive is or comprises one or more therapeutic agents. In general, a therapeutic agent is or comprises a small molecule and/or organic compound with pharmaceutical activity (e.g., activity that has been demonstrated with statistical significance in one or more relevant pre-clinical models or clinical settings). In some embodiments, a therapeutic agent is a clinically-used drug. In some embodiments, a therapeutic agent is or comprises an cells, proteins, peptides, nucleic acid analogues, nucleotides, oligonucleotides, nucleic acids (DNA, RNA, siRNA), peptide nucleic acids, aptamers, antibodies or fragments or portions thereof, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, hormones, hormone antagonists, growth factors or recombinant growth factors and fragments and variants thereof, cytokines, enzymes, antibiotics or antimicrobial compounds, antifungals, antivirals, toxins, prodrugs, chemotherapeutic agents, small molecules, drugs (e.g., drugs, dyes, amino acids, vitamins, antioxidants), pharmacologic agents, and combinations thereof

In some embodiments, the crosslinked zwitterionic gel comprises one or more cells. Cells suitable for use herein include, but are not limited to, progenitor cells or stem cells, smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

In some embodiments, the crosslinked zwitterionic gel comprise one or more antibiotics. Antibiotics suitable for incorporation in hydrogels include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, β-lactam antibiotics, rifamycins, novobiocin, fusidate sodium, capreomycin, colistimethate, gramicidin, doxycycline, erythromycin, nalidixic acid, and vancomycin. For example, β-lactam antibiotics can be aziocillin, aztreonam, carbenicillin, cefoperazone, ceftriaxone, cephaloridine, cephalothin, moxalactam, piperacillin, ticarcillin and combination thereof.

In some embodiments, the crosslinked zwitterionic gel comprises one or more anti-inflammatories. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

In some embodiments, the crosslinked zwitterionic gel comprises one or more antibodies. Suitable antibodies for incorporation in hydrogels include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

In some embodiments, the crosslinked zwitterionic gel comprises one or more polypeptides (e.g., proteins), including but are not limited to: one or more antigens, cytokines, hormones, chemokines, enzymes, and any combination thereof as an agent and/or functional group. Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

In some embodiments, the crosslinked zwitterionic gel comprises one or more therapeutic agents useful for wound healing. In some embodiments, agents useful for wound healing include stimulators, enhancers or positive mediators of the wound healing cascade which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

The amount of the therapeutic agent used in the radical crosslinked can and will vary depending on the specific therapeutic agent, the amount of the therapeutic that needs to be dosed per day, and the specific radical crosslinked zwitterionic gel used. Generally, the concentration percentage ratio of the therapeutic agent to the radical crosslinked zwitterionic gel may range from about 0.001:1.0 to about 1.0:1.0. In various embodiments, the concentration percentage ratio of the therapeutic agent to the radical crosslinked zwitterionic gel may range from about 0.001:1.0 to about 1.0:1.0, from about 0.01:1.0 to about 0.5:1.0, or from about 0.05:1.0 to about 0.1:1.0.

The radical crosslinked zwitterionic gel possess some novel and unique properties. The radical crosslinked zwitterionic possess an average pore size greater than about 50 mm and less than about 100 mm. In various embodiments, the average pore size D⁹° is about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, or about 99 mm. This pore size is important to allow for a timed release of the optional therapeutic.

The radical crosslinked zwitterionic gels are elastic, injectable, and compressible. After the compression is complete, the radical crosslinked zwitterionic gel releases and returns to its original form. Additionally, the radical crosslinked zwitterionic gels are self-healing. This term self-healing refers to removing a piece of the radical crosslinked zwitterionic gel from the main portion of the radical crosslinked zwitterionic gel, placing the removed piece onto the remaining main portion of the radical crosslinked zwitterionic, the removed piece merges into the main portion and reestablishes crosslinking.

Generally, the radical crosslinked zwitterionic gel has a storage modulus from about 50 Pa to about 2,000 Pa. In various embodiments, the radical crosslinked zwitterionic gel has a storage modulus from 50 PA to about 2,000 Pa, from about 100 Pa to about 1,800 Pa, from about 200 to about 1,700 Pa, from about 300 to about 1,600 Pa, from about 400 to about 1,500 Pa, from about 500 to about 1,400 Pa, from about 600 to about 1,300 Pa, from about 700 to about 1,200 Pa, from about 800 to about 1,100 Pa, from about 900 to about 1,000 Pa, or from about 500 Pa to about 750 Pa.

In general, the radical crosslinked zwitterionic gel has an elastic modulus from about 1,000 Pa to about 20,000 Pa. In various embodiments, the radical crosslinked zwitterionic gel has an elastic modulus from about 1,000 Pa to about 20,000 Pa, from about 2,000 Pa to about 17,250 Pa, from about 2,500 Pa to about 15,000 Pa, from about 3,000 Pa to about 12,500 Pa, from about 3,500 Pa to about 10,000 Pa, from about 4,000 Pa to about 7,500 Pa, or from about 5,000 Pa to about 10,000 Pa.

The physical properties of the radical crosslinked zwitterionic gels of the disclosure provide hydrogel materials that are anti-fouling, sticky, self-healing, cytocompatible, and provide improved healing of a wound. In certain aspects, the self-healing properties of the radical crosslinked zwitterionic gels of the disclosure facilitate the ability of the gel to conform to the size and shape of a wound.

Table 1 provides the effects of crosslinking time with different embodiments of the invention.

TABLE 1

| | Polymer | Monomers | | Crosslinking time | |
|---|---|---|---|---|---|
| Sample | Content (w/w) | Content (w/w) | Photoinitiator | (405 nm LED) | Results |
| 1A | 25 wt. % | 30% SBMA: | 0.05 v % PEGDM | 10 mins. | Good |
| | | 70% HEMA | | | |
| 2A | 25 wt. % | 30% SBMA: | 0.05 v % PEGDM | 30 mins. | Poor |
| | | 70% HEMA | 0.05 wt % LAP | | |
| 3A | 25 wt. % | 30% SBMA: | 0.05 v % PEGDM | 60 mins. | Poor |
| | | 70% HEMA | 0.05 wt % LAP | | |
| 4A | 25 wt. % | 30% SBMA: | 0.05 wt % LAP | 60 mins. | Good |
| | | 70% HEMA | | | |
| 1B | 50 wt. % | 30% SBMA: | 0.05 v % PEGDM | 10 mins. | Good |
| | | 70% HEMA | 0.05 wt % LAP | | |
| 2B | 50 wt. % | 30% SBMA: | 0.05 v % PEGDM | 30 mins. | Good |
| | | 70% HEMA | 0.05 wt % LAP | | |
| 3B | 50 wt. % | 30% SBMA: | 0.05 wt % LAP | 20 mins. | Good |
| | | 70% HEMA | | | |
| 4B | 50 wt. % | 30% SBMA: | 0.05 v % PEGDM | 20 mins. | Poor |
| | | 70% HEMA | | | |

LAP—Lithium phenyl-2,4,6-trimethylbenzoylphosphinate
PEGDM—Poly(ethylene glycol) dimethacrylate

(II) Methods for Preparing Radical Crosslinked Zwitterionic Gels

In another aspect, the present disclosure provides for methods to prepare the radical crosslinked zwitterionic gel. The methods comprise: (a) mixing a zwitterionic monomer, a non-zwitterionic ionic monomer, an optional cross linking agent, an optional rheology modifier, an optional antioxidant, and a solvent (e.g., water) to form a reaction mixture; (b) optionally filtering the reaction mixture through a particle filter, e.g., a 0.22 μm pore filter to thereby form a filtered reaction mixture; and (c) exposing the reaction mixture and/or filtered reaction mixture to light and/or other suitable radical initiator thereby polymerize the zwitterionic monomer and the non-zwitterionic ionic monomer to thereby form a radical crosslinked zwitterionic gel, wherein the radical crosslinked zwitterionic gel has an average pore size greater than about 50 mm and less than about 100 mm. In some embodiments, the preparation methods of the disclosure may further comprise washing the radical crosslinked zwitterionic gel to remove unreacted monomers, crosslinking agents, and optional initiator by-products (if present), to thereby form a washed radical crosslinked zwitterionic gel.

The zwitterionic monomer, the non-zwitterionic monomer, and optional crosslinking agents are detailed above. In general, the reaction mixture and/or filtered reaction mixture may be mixed for a duration of time sufficient to provide a homogeneous solution. Various methods of mixing are known in the art. In certain embodiments, the reaction mixture and/or filtered reaction mixture may be mixed prior to exposure to light or other suitable radical generator, or the reaction mixture and/or filtered reaction mixture may be mixed contemporaneously with exposure to light or other suitable radical generator.

In one embodiment, the zwitterionic monomer is 2-(methacryloyloxy) ethyl] dimethyl-(3-sulfopropyl)ammonium hydroxide (SBMA); the non-zwitterionic monomer is hydroxyethyl methacrylate (HEMA); and the crosslinking agent is optionally polyethylene glycol dimethacrylate (PEGDMA).

In general, the weight ratio of the zwitterionic monomer to the non-zwitterionic monomer in the initial reaction mixture may range from about 1:19 to about 19:1. In various embodiments, the weight ratio of the zwitterionic monomer to the non-zwitterionic monomer in the initial reaction mixture may range from 1:19 to about 19:1, from about 1:9 to about 9:1, from about 1:4 to about 4:1, or from about 2:1 to about 1:2.

Generally, the ratio of concentration percentages of the crosslinking agent initially present in the reaction mixture to the radical crosslinked zwitterionic gel formed may range from about 0.0:1.0 to about 0.25:1.0. In various embodiments, the concentration percentage ratio of the crosslinking agent initially present in the reaction mixture to the radical crosslinked zwitterionic gel form may range from about 0.0:1.0 to about 0.25:1.0, from about 0.001:1.0 to about 0.2:1.0, from about 0.005:1.0 to about 0.15:1.0, from about 0.01:1.0 to about 0.1:1.0.

In general, the volume of the solvent (e.g., water) to the weight of the zwitterionic monomer, the non-zwitterionic monomer, and the optional crosslinking agent present in the initial reaction mixture may range from about 0.1:1.0 to about 500.0:1.0. In various embodiments, the volume of the solvent to the weight of the zwitterionic monomer, the non-zwitterionic monomer, and the optional crosslinking agent in the initial reaction mixture may range from about 0.1:1.0 to about 500.0:1.0, from about 1.0:1.0 to about 250.0:1.0, or from about 5.0:1,0 to about 100.0:1.0.

The reaction mixture may optionally comprise an initiator. Without being limited by theory, the initiator may facilitate the polymerization between the zwitterionic monomer, the non-zwitterionic monomer, and the optional crosslinking agent. By way of non-limiting example, initiators useful in connection with the preparation methods of the disclosure include sodium persulfate, ruthenium(II)-tris(2,2′-bipyridyl) dichloride, and a combination thereof.

In general, the weight ratio of the initiator to the weight ratio of the zwitterionic monomer, non-zwitterionic monomer, and the optional crosslinking agent in the initial reaction mixture may range from 0.001:1.0 to 0.1:1.0. In various embodiments, the weight ratio of the initiator to the weight ratio of the zwitterionic monomer, non-zwitterionic monomer, and the optional crosslinking agent in the initial reaction mixture may range from 0.001:1.0 to 0.1:1.0, from about 0.005:1.0 to about 0.05:1.0, or from about 0.01:1.0 to about 0.02:1.0.

Once formed, the reaction mixture may optionally be filtered to reduce particle size, e.g., through a 0.22 μm pore filter, to thereby form a filtered reaction mixture. Without intending to be limited by theory, filtration may remove undissolved materials from the reaction mixture, facilitate formation of a homogeneous reaction mixture, and allow for polymerization to occur more efficiently.

The reaction mixture and/or filtered reaction mixture may then be exposed to light and/or other suitable radical generator to initiate the polymerization. Without intending to be limited by theory, light and/or radial generator initiation allows the zwitterionic monomer, the non-zwitterionic monomer, optional crosslinking agent, and optional rheology modifier to form a radical crosslinked zwitterionic gel. The optional initiator may further enhance the crosslinking polymerization.

Various forms of light and/or radical generators can be used to polymerize the reaction mixture. Wavelengths of light useful in generating a radical generally range from 400 nm to 700 nm. Non-limiting examples of useful forms of light may be UV light, visible light, or white light, e.g., 405 nm. The light may be sunlight, filtered sunlight, or from an artificial source (e.g. incandescent light, LED light, UV light, etc.).

Other suitable radical generators may be used alone or in combination with light exposure. Non-limiting examples of suitable radical generators may be ammonium persulfate (APS), N,N,N′N′-tetramethylethylene-1,2-diamine (TEMED), ascorbic acid, organic peroxides, and combinations thereof. In some embodiments, suitable radical generators may include APS/TEMED or APS/ascorbic acid (e.g., 10 mM ascorbic acid, 10 mM APS). Other thermal and non-thermal initiators known in the art may also be used. In certain embodiments, non-thermal radical generators may release heat during reaction, and may require cooling. By way of example, K₂S₂O₈ (decomposition temperature of 50-60 degrees C.) or 4,4′-azobis(4-cyanovaleric acid) (decomposition of 70 degrees C.) may be used. Table 2 illustrates the effects of radical scavenger concentration on gel stiffness. Table 2 illustrates the effects of varying concentration of ammonium persulfate (APS) on gel toughness and adhesiveness.

TABLE 2

| | Polymer | Monomers | | Toughness and |
|---|---|---|---|---|
| APS Concentration¹ | Content (w/w) | Content (w/w) | Conditions | Adhesiveness |
| 1.6 mg/mL | 25 wt. % | 30% SBMA: | 1 hour - Room temp. | **** |
| | | 70% HEMA | | |
| 0.8 mg/mL | 25 wt. % | 30% SBMA: | 1 hour - Room temp. | *** |
| | | 70% HEMA | | |
| 0.4 mg/mL | 25 wt. % | 30% SBMA: | 1 hour - Room temp. | ** |
| | | 70% HEMA | | |
| 0.2 mg/mL | 25 wt. % | 30% SBMA: | 1 hour - Room temp. | * |
| | | 70% HEMA | | |

¹All mixtures also included TEMED in a constant amount.
* Increasing APS concentration increased gel toughness and adhesiveness

Without intending to be limited by theory, the use of ascorbic acid as a free radical activator in in situ polymerization opens a possibility to eliminate harmful substances, e.g., from ceramic slurries. Ascorbic acid (one of its form is commonly known as vitamin C) is a naturally occurring compound, which can be found in single-cell organisms, plants and animals, where it is produced from glucose. It dissolves well in water and is not harmful for humans and environment. Moreover, due to its mild acidic properties, it can positively influence the stability of ceramic suspensions, while the addition of N,N,N′,N′-tetramethylethylenediamine, which is an alkaline amine, leads to the coagulation of some ceramic slurries. Table 3 illustrates the effects of radical scavenger concentration on gel stiffness.

TABLE 3

| Radical Scavenger | Polymer | Monomers | | |
|---|---|---|---|---|
| (concentration (mM)) | Content (w/w) | Content (w/w) | Photo-Crosslinked | Stiffness |
| Ascorbic Acid (0) | 25 wt. % | 30% SBMA: | 1 hour - 405 nm | **** |
| | | 70% HEMA | | |
| Ascorbic Acid (2 mM) | 25 wt. % | 30% SBMA: | 1 hour - 405 nm | *** |
| | | 70% HEMA | | |
| Ascorbic Acid (4 mM) | 25 wt. % | 30% SBMA: | 1 hour - 405 nm | ** |
| | | 70% HEMA | | |
| Ascorbic Acid (8 mM)) | 25 wt. % | 30% SBMA: | 1 hour - 405 nm | * |
| | | 70% HEMA | | |

* decreasing gel stiffness with increased radical scavenger concentration

The reaction mixture may be placed into a suitable receptacle and exposed to the light source and/or other suitable radical generator. In certain embodiments, the reaction receptacle may be configured to allow for the specific wavelength of light to contact the reaction mixture to provide polymerization. The duration of the exposing the reaction mixture to light and/or other suitable radical generator may vary depending on the amounts of the monomers, optional crosslinking agent, optional initiator, optional rheology modifier, the types of monomers used, and the optional antioxidant used. In general, the duration of exposing the reaction mixture to light and/or other suitable radical generator can range from about 1 minute to 24 hours. In various embodiments, the duration of exposure may range from about 30 minutes to 24 hours, from about 45 minutes to 6 hours, or from about 1 hour to 2 hours. In one embodiment, the duration of exposure is about 1 hour.

In certain embodiments, the radical crosslinked zwitterionic gel may be washed, e.g., with a buffered saline solution, to thereby remove unreacted monomers, unreacted crosslinking agent, and optional initiator by-products, to thereby form a washed radical crosslinked zwitterionic gel.

In general, the radical crosslinked zwitterionic gel may be washed at least once with the buffered saline solution. In various embodiments, the radical crosslinked zwitterionic gel may be washed once, twice, or more than twice with the buffered saline solution. Each wash of the radical crosslinked zwitterionic gel not only removes the unreacted material but also increases the purity. Each wash of the buffered saline solution may be incubated with the radical crosslinked zwitterionic gel for a period of time to allow the unreacted monomers, unreacted crosslinking agent, and initiator by-products to diffuse out of the gel.

Generally, a wide variety of buffered saline solutions may be used to wash the radical crosslinked zwitterionic gels. Representative examples of suitable buffering salines include, but are not limited to phosphates, carbonates, citrates, tris buffers, and buffered saline salts. In one embodiment, the buffered saline useful in the method is phosphate buffered saline.

The amount of the buffered saline solution may vary depending on the preparation size of the radical crosslinked zwitterionic gel, the type of monomers and optional crosslinking agent, the amount of the optional initiator, and the buffered saline used. Generally, the volume of buffered saline solution to the weights of the zwitterionic monomer, the non-zwitterionic monomer, the optional crosslinking agent, and the amount of the initiator in the initial reaction mixture may range from about 1.0:1.0 to about 100:1.0. In various embodiments, the volume of buffered saline solution to the weights of the zwitterionic monomer, the non-zwitterionic monomer, and the optional crosslinking agent in the initial reaction mixture may range from about 1.0:1.0 to about 100:1.0, from about 5.0:1.0 to about 90.0:1.0, from about 10.0:1.0 to about 50.0:1.0, or from about 15:1.0 to about 30.0:1.0.

Generally, the preparation methods of disclosure may be conducted at a temperature that ranges from 0° C. to about 40° C. In various embodiments, the preparation methods may be conducted at a temperature that ranges from 0° C. to about 40° C., from about 10° C. to about 30° C., or from about 15° C. to about 25° C. In one embodiment, the preparation methods may be conducted at room temperature (e.g., —23° C.). The preparation methods of the disclosure are typically performed under ambient pressure and may also be conducted under an inert atmosphere, for example under nitrogen, argon or helium. The radical crosslinked zwitterionic gels prepared according to the methods disclosed herein may be stored at 4° C. before use.

The radical crosslinked zwitterionic gel may be further lyophilized to prepare a dry power of the radical crosslinked zwitterionic gel. This lyophilization method allows for a convenient powder which can be re-hydrolyzed before use. In this form, the radical crosslinked zwitterionic gel has increased storage life.

In some embodiments the radical crosslinked zwitterionic gel may be dissolved in a solvent prior to end use, followed by precipitation using a co-solvent. Without intending to be limited by theory, residual initiators and stabilizers will generally remain in solution and the polymers will separate out as a solid (powder, gum or fibers). This process may be repeated until desirable polymer characteristics are obtained. This fractional precipitation is also effective in removing lower molecular weight polymers, resulting in narrower molecular weight distribution. Solvent/co-solvent pairs that may be used include Toluene/hexane, toluene/methanol, THF/water, etc.

Additional aspects of the preparation methods of the disclosure are shown in the appendix.

(III) Methods of Treating a Wound

In another aspect, the present disclosure encompasses methods of treating a wound or providing for wound healing. The methods comprise administering or applying the radical crosslinked zwitterionic gel (or washed radical crosslinked zwitterionic gel) described herein to a wound of a subject in need thereof, and leaving the gel in place for an amount of time sufficient to provide for wound healing. In certain embodiments, the wound healing provided in accordance with the methods of the disclosure may be partial wound healing (e.g., wound closure) or complete wound healing (e.g., wound closure). In certain embodiments, the wound may be at least 10%, at least 20%, at least 25%, at least 30%, at least 40%, at least 50%, at least 60%, at least 70%, at least 75%, at least 80%, at least 90%, at least 95%, at least 98%, at least 99%, or completely healed (e.g., closed), as compared to wound prior to treatment.

In certain embodiments, the radical crosslinked zwitterionic gel (or washed radical crosslinked zwitterionic gel) may be topically applied to the surface of a wound, or may be injected into a wound. The radical crosslinked zwitterionic gel may be applied to various sizes and shapes of wounds. In certain embodiments, the wound may be a diabetic ulcer. In other embodiments, the wound may be a surgical or trauma wound. One or more applications of the radical crosslinked zwitterionic gel may be applied over an extended period of time.

The radical crosslinked zwitterionic gel may be applied in various means to the wound. Non-limiting methods may be by a syringe, applying the radical crosslinked zwitterionic gel using a tongue depressor, or applying the radical crosslinked zwitterionic gels to a bandage or a band aid then applying the bandage to the wound. The lyophilized radical crosslinked zwitterionic gel can be readily re-hydrated and applied directly to the wound.

A subject that may be treated according to the methods of the disclosure include a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

Additional aspects of the therapeutic methods of the disclosure are shown in the appendix.

As various changes could be made in the above-described methods without departing from the scope of the invention, it is intended that all matter contained in the above description and in the examples given below, shall be interpreted as illustrative and not in a limiting sense.

### EXAMPLES

Materials:

[2-(Methacryloyloxy) ethyl]dimethyl-(3-sulfopropyl)ammonium hydroxide (SBMA) was purchased from AFG Scientific (Northbrook, Ill.) and 3-[[2-(methacryloyloxy)ethyl]dimethylammonio]propionate (CBMA) was purchased from TCI Chemicals (Portland, Oreg.). Hydroxyethyl methacrylate (HEMA), sodium persulfate (SPS), polyethylene glycol dimethacrylate (PEGDMA), and ruthenium (II) tris-bipyridyl (Ru (bpy)) dichloride were purchased from Sigma Aldrich (St. Louis, Mo.).

### Example 1: Preparation of Radical Crosslinked Zwitterionic Gels using White Light (More Detailed Procedures are Needed)

CBMA, SBMA, and HEMA were used as received without any further processing. PEGDMA was diluted to 10% v/v in water, SPS and Ru(bpy) were dissolved at 10 mg/mL. The desired dry weight of zwitterionic monomer was added to a 1.5 mL microcentrifuge tube. Then, varying volumes of HEMA, SPS, Ru (bpy), and PEGDMA were added to the tube. The total volume of the solution was then brought up to 1 mL with water, and the solution was vortexed for 15 seconds to fully dissolve the zwitterionic monomer. The solution was then sterile filtered through a 0.22 μm pore filter. The tube was then placed under ambient, white light for 1 hour to induce polymerization. Following polymerization, the gels were rinsed three times with sterile PBS and stored at 4° C. Table 4 shows the amount of the monomers and optional crosslinking agent and the results from these experiments:

TABLE 4

| | PEGDMA | SBMA | HEMA | Description |
|---|---|---|---|---|
| 20% | 0.1% | 10% | 90% | Loose, malleable gel, slightly |
| Polymer | | | | opaque |
| | | 25% | 75% | Very viscous liquid, slightly |
| | | | | gelled |
| | | 50% | 50% | No gel |
| | 0.5% | 10% | 90% | Cohesive, brittle, translucent gel |
| | | 25% | 75% | Cohesive, sticky, brittle gel |
| | | 50% | 50% | Cohesive, sticky, brittle gel |
| | 1% | 10% | 90% | No gel |
| | | 25% | 75% | Partial gel, mostly liquid |
| | | 50% | 50% | Viscous, cohesive gel |
| | 3% | 10% | 90% | Cohesive Gel |
| | | 25% | 75% | Cohesive gel, slightly brittle |
| | | 50% | 50% | Cohesive, brittle gel |
| | 5% | 10% | 90% | Firm, cloudy gel |
| | | 25% | 75% | Stiff, springy gel |
| | | 50% | 50% | Stiff, brittle gel |
| 25% | 0.1% | 10% | 90% | No gel |
| Polymer | | 20% | 80% | No gel |
| | | 30% | 70% | No gel |
| | 0.5% | 10% | 90% | No gel |
| | | 20% | 80% | No gel |
| | | 30% | 70% | Flowing, goopy, sticky liquid |
| | | 40% | 60% | Injectable, sticky, viscous gel |
| | 1% | 10% | 90% | Viscous, sticky liquid |
| | | 20% | 80% | Very viscous, goopy, sticky |
| | | | | liquid |
| | | 30% | 70% | Near-solid, goopy, sticky |
| | 0.1% | 10% | 90% | No gel |
| | | 25% | 75% | No gel |
| | | 50% | 50% | No gel |
| 30% | 0.5% | 10% | 90% | No gel |
| Polymer | | 25% | 75% | Stretchy, viscous liquid |
| | | 50% | 50% | Viscous, gooey liquid |
| | 1% | 10% | 90% | Viscous, gooey liquid |
| | | 25% | 75% | Sticky, semi-brittle gel |
| | | 50% | 50% | Brittle, cohesive gel |

### Example 2: Preparation of Radical Crosslinked Zwitterionic Gels using 405 nm Light

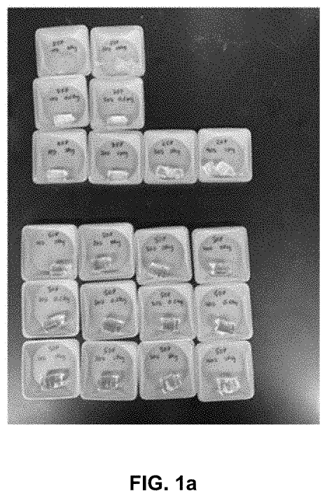

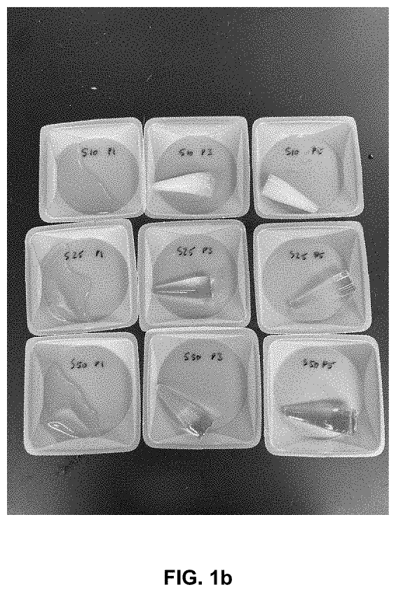

CBMA, SBMA, and HEMA were used as received without any further processing. The desired dry weight of zwitterionic monomer was added to a 1.5 mL microcentrifuge tube along with the desired volume of HEMA. The total solution volume was then brought up to 1 mL with water. The solution was vortexed for 15 seconds to dissolve all components and sterile filtered through a 0.22 μm pore filter. Then, the solution was placed into a custom light box with near-UV, 405 nm light for 1 hour. After polymerization, the gels were rinsed with sterile PBS and stored at 4° C. Table 5 shows the amount of the monomers and optional crosslinking agent and the results from these experiments. FIGS. 1a and 1b illustrates photographs of the radical crosslinked zwitterionic gels.

TABLE 5

| Polymer | | PEGDMA | |
|---|---|---|---|
| Concentration | SBMA:HEMA | Conc (%) | Description |
| 25% | 10:90 | 0 | Flowable, sticky, loose, injectable gel |
| | | 0.5 | Slightly opaque, slippery, soft, squishy gel pellet |
| | | 1 | Slightly opaque, slippery, squishy gel pellet |
| | 20:80 | 0 | Clear, squishy gel pellet |
| | | 0.5 | Clear, slippery, squishy gel pellet |
| | | 1 | Slightly opaque, slippery, stiff gel |
| | 30:70 | 0 | Clear, Sticky, Moldable Gel, |
| | | 0.5 | Maintains Shape, Cannot Self Heal, Very Stiff |
| | | 1 | Stiff gel pellet |
| | 40:60 | 0 | Clear, Sticky, Moldable Gel, |
| | | 0.5 | Maintains Shape, Cannot Self Heal, Very Stiff |
| | | 1 | Brittle, stiff gel pellet |
| 50% | 10:90 | 0 | Soft, squishy, sticky gel pellet |
| | | 0.5 | Stiff gel pellet |
| | | 1 | Stiff gel pellet |
| | 20:80 | 0 | Stiff gel pellet |
| | | 0.5 | Stiff gel pellet |
| | | 1 | Stiff gel pellet |
| | 30:70 | 0 | Stiff gel pellet |
| | | 0.5 | Stiff gel pellet |
| | | 1 | Stiff gel pellet |
| | 40:60 | 0 | Stiff gel pellet |
| | | 0.5 | Stiff gel pellet |
| | | 1 | Stiff gel pellet |

### Example 3: Cytocompatability of Radical Crosslinked Zwitterionic Gels

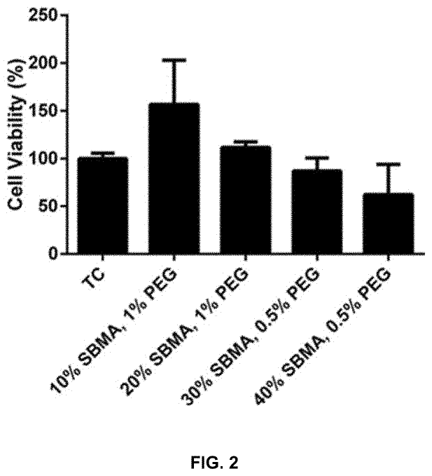

Gels were fabricated according to the protocol described above. The gels were then aliquoted into transwell membranes with 0.4 μm pore size in a 24-well plate. To rinse excess unreacted monomer and reagents, the gels were submerged in sterile 1× PBS (pH 7.4) for three days. Each day, PBS was aspirated and replaced with fresh solution. Following the rinse steps, 50,000 human dermal fibroblast cells (hDFs) were seeded into a tissue culture (TC) treated 24 well plate, and the transwell membranes containing the gels were placed on top of the culture. The culture was incubated with humidity at 37° C. and 5% CO₂ for 24 hours. To test cell viability after 24 hours, 50 μL of CCK8 (Dojindo) was added to the 500 μL of cell culture media and incubated for one hour, according to manufacturer's protocol. Then, 100 μL of the media was transferred to a 96-well plate (n=3) and the absorbance was read with a microplate reader at 450 nm. FIG. 2 shows the results of these experiments.

### Example 4: Antifouling of Radical Crosslinked Zwitterionic Gels

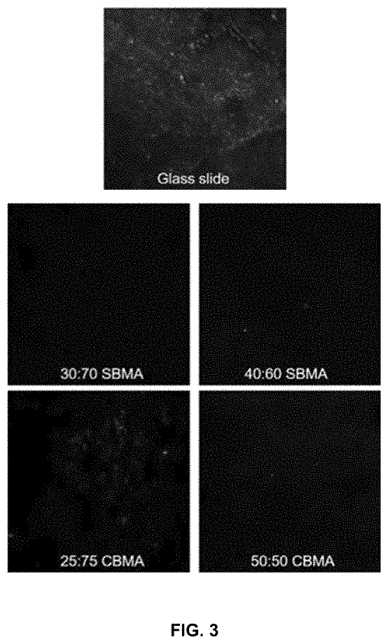

Anti-fouling capabilities of the zwitterionic gels was measured using fluorescent microscopy. Circular borosilicate glass cover slips were rinsed with absolute ethanol and then desiccated for 10 minutes to dry. The glass slides were then transferred to a 24 well plate and covered with 200 μL of zwitterionic gel solution. The gels were polymerized for one hour, after which the slides were transferred to an unused well. Then, 200 μL of FITC-BSA solution (0.2 mg/mL) was added to each glass slide and the slides were shaken briefly to ensure full coverage of the protein solution. The plate was then covered in foil to protect it from light, and incubated overnight at 37° C. The following day, the slides were imaged using a Lecia fluorescent microscope with λₑₓ=495 nm and λₑₘ=520 nm. FIG. 3 shows photographs of this antifouling behavior as a comparison to a glass slide.

### Example 5: Rheology Measurements of Radical crosslinked Zwitterionic Gels

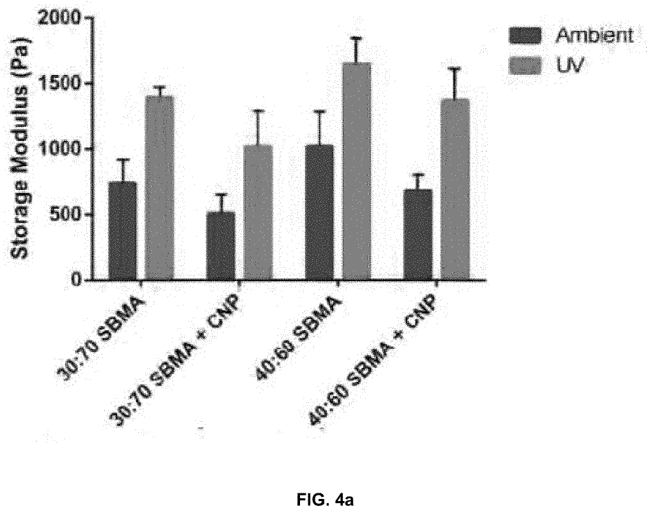

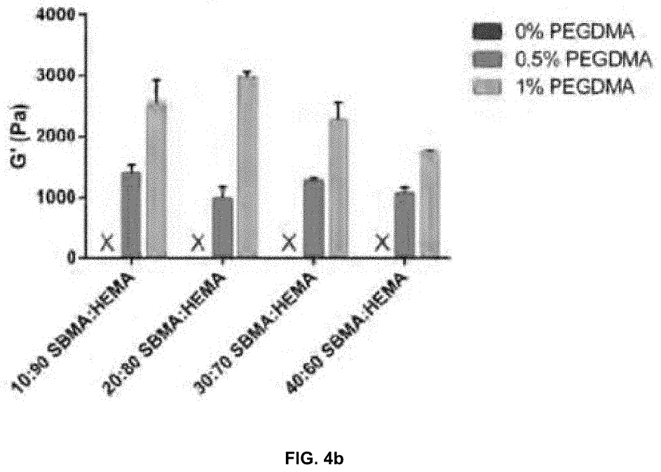

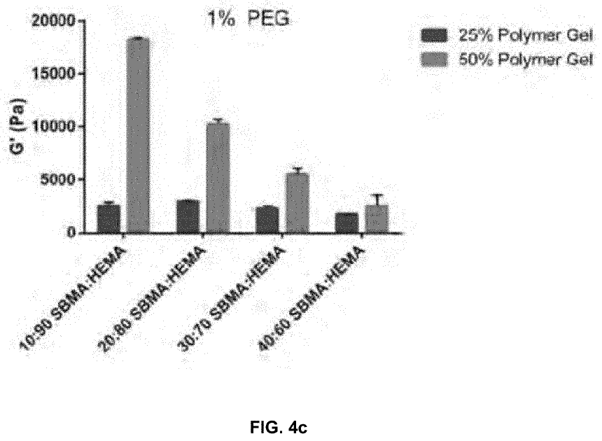

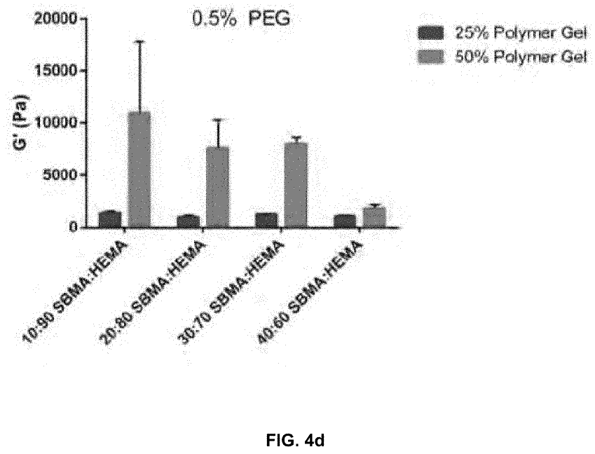

Gel samples of about 22 mm diameters and about 4 mm in height were prepared in 12 well plates. To determine the rheological properties of the gels, frequency sweep and strain sweeps test were performed using an AR-G2 rheometer (TA Instruments) equipped with a 20 mm diameter crosshatched parallel plate at 37° C. Frequency sweep measurements were performed at 1% strain. Strain sweep measurements were performed at 10 rad/s frequency. For FTIR measurements gel samples were lyophilized and dried powders were analyzed using a Nexus 470 ESP FT-IR Spectrometer equipped with an ATR accessory (Specac, Golden Gate). FIG. 4a-4d illustrates the results from these measurements.

### Example 6: Swelling Tests of Radical Crosslinked Zwitterionic Gels

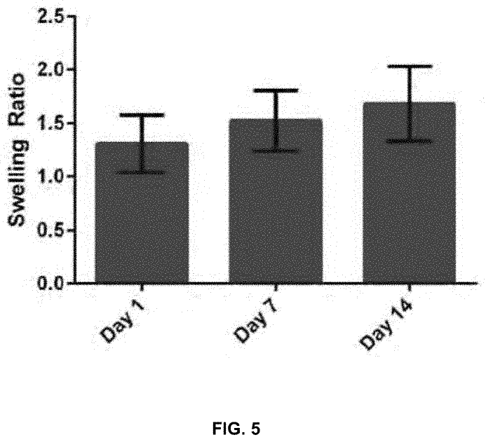

To determine the swelling behavior of the radical zwitterionic hydrogel, as-prepared gels were weighed to determine the mass of initial samples (mᵢₙᵢₜᵢₐₗ), soaked in PBS (pH:7.4), and allowed to swell in an incubator at 37° C. The hydrogels were taken at selected time intervals and transferred from one petri dish to another several times to remove excess water from the hydrogel surface, and then weighed (m_{wet gel}) to determine the swelling of the gels. FIG. 5 illustrates the swelling ratio of the radical crosslinked zwitterionic gels.

### Example 7: Degradation Tests of Radical Crosslinked Zwitterionic Gels

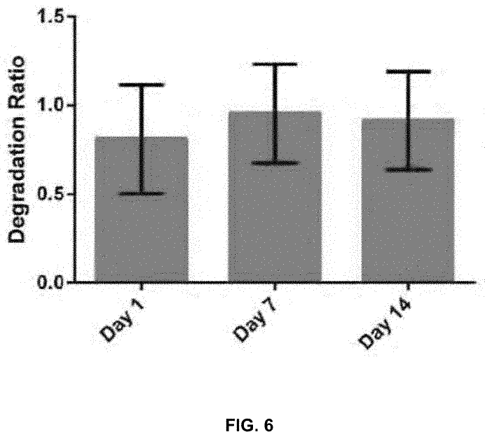

To determine the degradation behavior of the hydrogels, gels were soaked in PBS (pH 7.4) and allowed to sit in an incubator at 37° C. The hydrogels were taken at selected time intervals, lyophilized, and weighed to determine the degradation percentage at different time intervals. FIG. 6 illustrates the degradation ratio of the radical crosslinked zwitterionic gels.

### Example 8: Injectability, Stretchability, Compression and Release, and Self-Healing of Radical Ccrosslinked Zwitterionic Gels

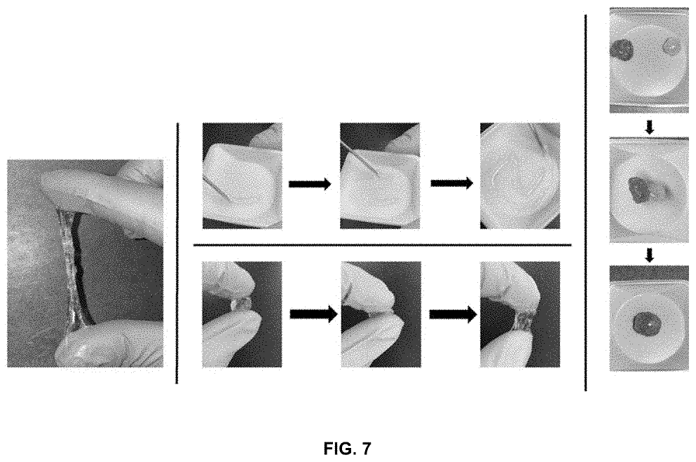

FIG. 7 shows a series of photographs on the injectability, stretchability, compression and release, and self-healing characteristics of the radical crosslinked zwitterionic gels prepared in white light.

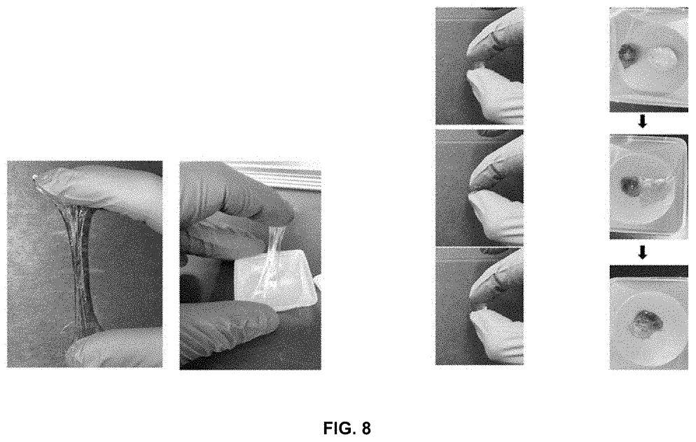

FIG. 8 shows a series of photographs on the injectability, stretchability, compression and release, and self-healing characteristics of the radical crosslinked zwitterionic gels prepared in 405 nm light.

### Example 9: Injectability of Radical Crosslinked Zwitterionic Gels

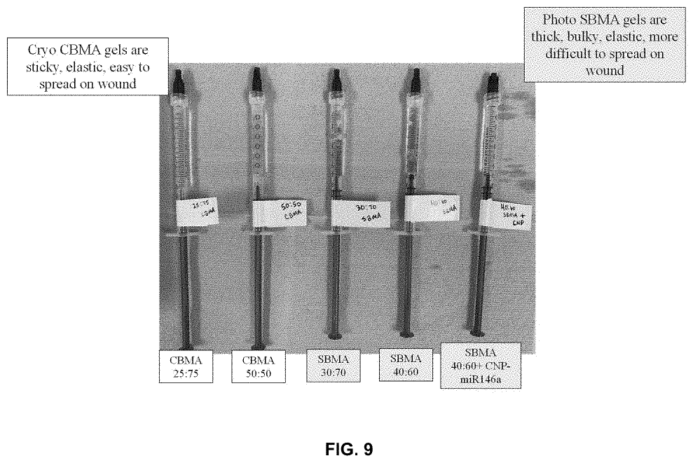

FIG. 9 shows a photograph on the injectability characteristic of the radical cross linked zwitterionic gels versus zwitterionic gels prepared using the cryogenic process.

### Example 10: Sustained Release of Therapeutics

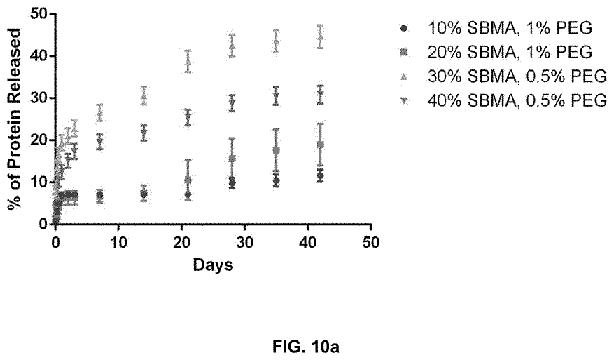

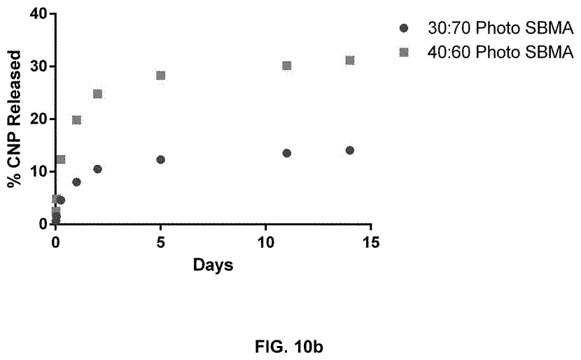

FIGS. 10a and 10b are graphs which show release of myoglobin and CNP of various radical crosslinked zwitterionic gels over an extended period of time.

### Example 11: Wound Closure Experiments

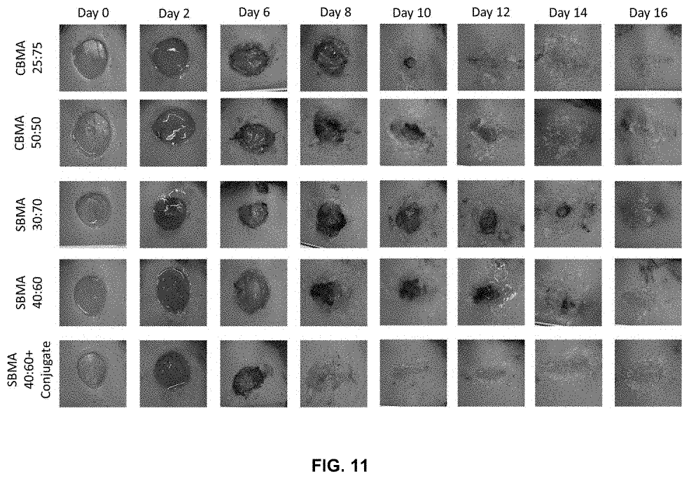

The radical crosslinked zwitterionic gels were evaluated on closing wounds in a human. (Need more details). FIG. 11 are a series of photographs showing the radical crosslinked zwitterionic gels, applied to a wound, enhances healing over a period of 16 days.

### Example 12: Wound Closure Experiments in Mouse Study

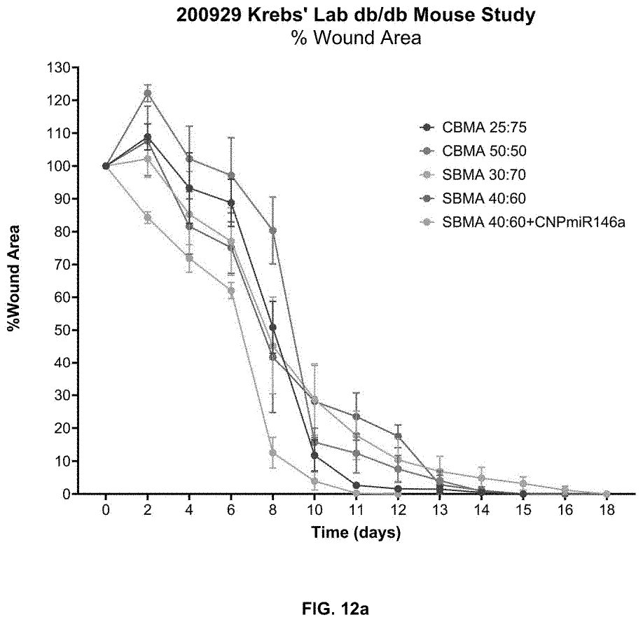

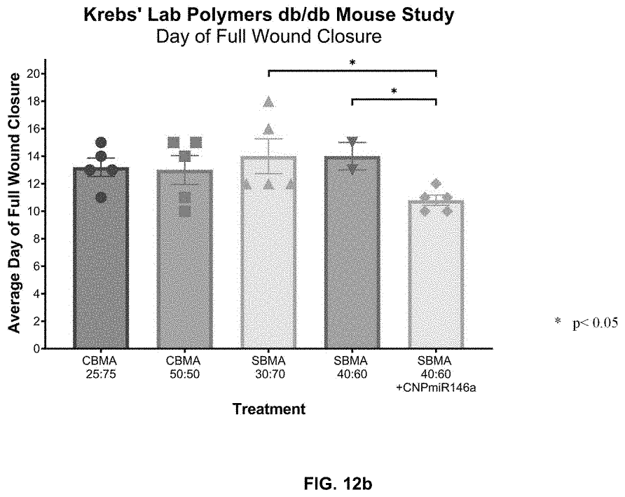

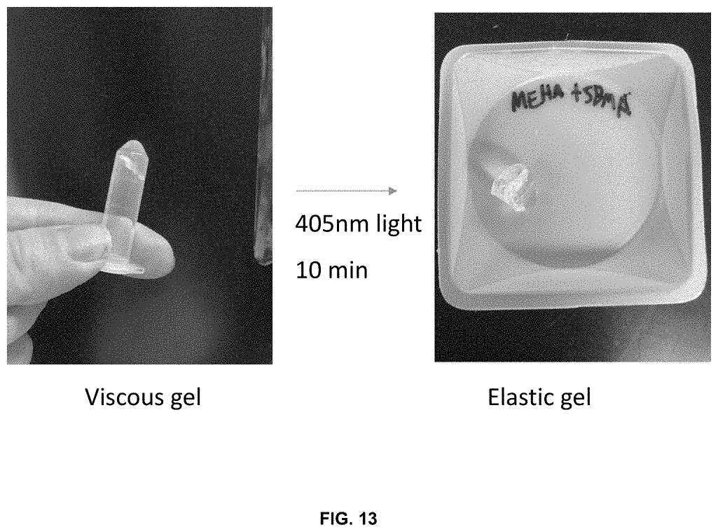

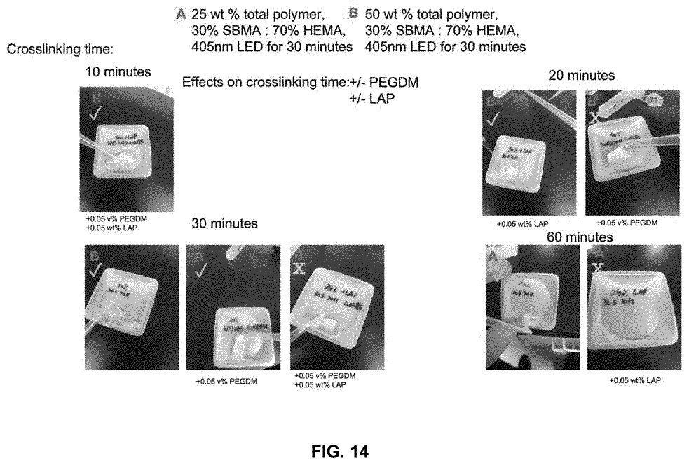

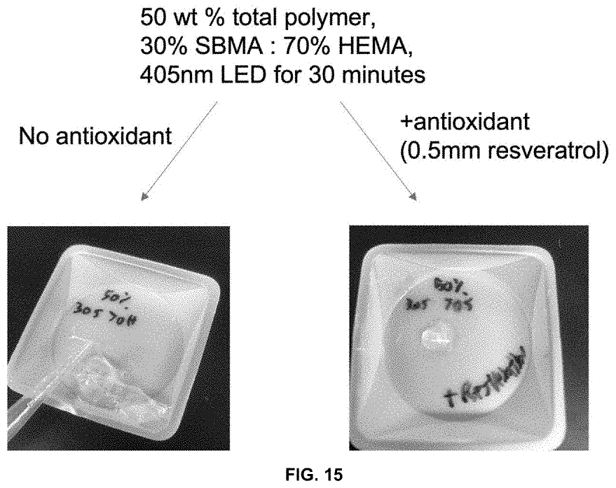

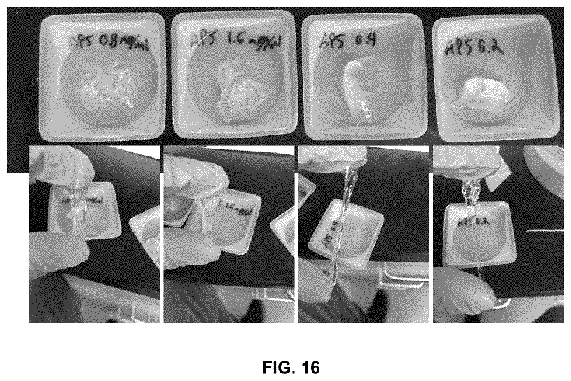

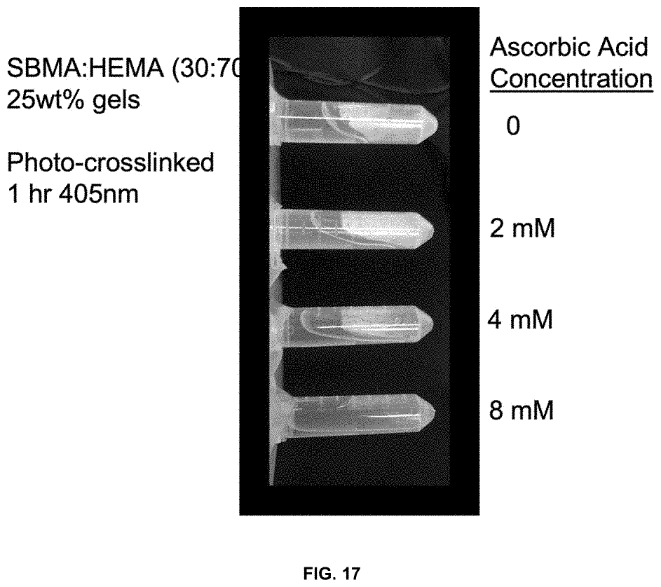

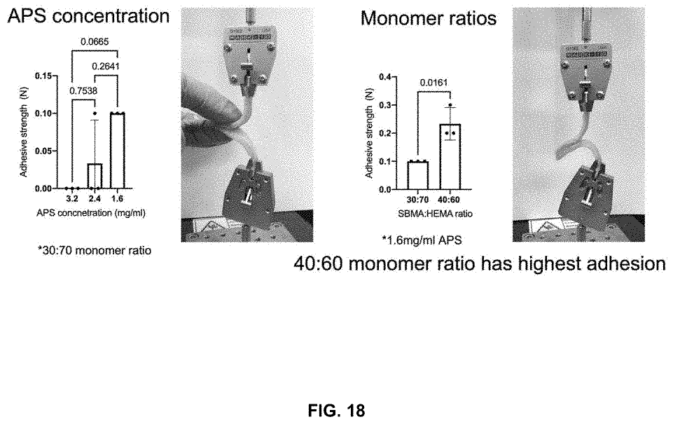

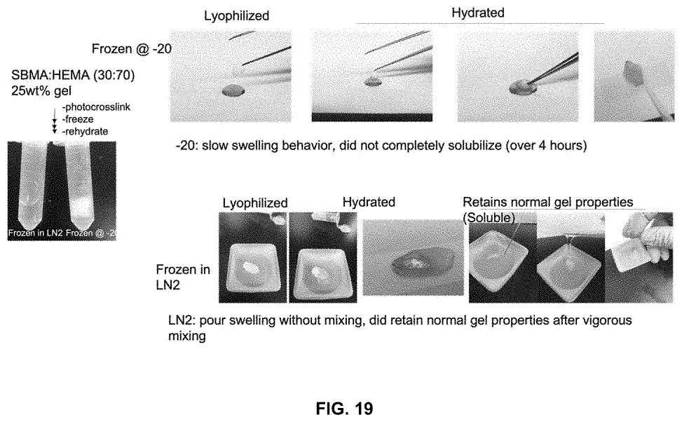

The radical crosslinked zwitterionic gels were applied to a series of mice to determine the percentage of wound closure over a period of days. As the data indicates, the wounds were fully closed in a 2-week period. FIGS. 12a and 12b shows graphs of% wound closure as measured versus time.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

1. A radical crosslinked zwitterionic gel, the radical crosslinked zwitterionic gel comprising:
a zwitterionic monomer selected from the group consisting of [2-(methacryloyloxy) ethyl]dimethyl-(3-sulfopropyl)ammonium hydroxide (SBMA), [(methacryloyloxy)ethyl] dimethylammonio]propionate (CBMA), 2-Methacryloyloxyethyl phosphorylcholine (MPC), and combinations thereof; and
a non-zwitterionic monomer.

2. The radical crosslinked zwitterionic gel of claim 1, wherein the non-zwitterionic monomer is hydroxyethyl methacrylate (HEMA).

3. The radical crosslinked zwitterionic gel of claim 1, further comprising a crosslinking agent.

4. The radical crosslinked zwitterionic gel of claim 3, wherein the crosslinking agent is selected from a group consisting of glycerol dimethacrylate (GDMA), methylene bisacrylamide (MBA), polyethylene glycol dimethacrylate (PEGDMA), and a combination of two or more crosslinkers.

5. The radical crosslinked zwitterionic gel of claim 4, wherein the crosslinking agent is polyethylene glycol dimethacrylate (PEGDMA).

6. The radical crosslinked zwitterionic gel of claim 1, further comprising an antioxidant. The radical crosslinked zwitterionic gel of claim 6, wherein the antioxidant is resveratrol.

8. The radical crosslinked zwitterionic gel of claim 1, further comprising a rheology modifier.

9. The radical crosslinked zwitterionic gel of claim 8, wherein the rheology modifier is a clay.

10. The radical crosslinked zwitterionic gel of claim 1, further comprising a therapeutic agent.

11. The radical crosslinked zwitterionic gel of claim 1, wherein the radical crosslinked zwitterionic gel is elastic.

12. The radical crosslinked zwitterionic gel of claim 1, wherein the radical crosslinked zwitterionic gel is injectable.

13. The radical crosslinked zwitterionic gel of claim 1, wherein the radical crosslinked zwitterionic is compressible and releasable.

14. The radical crosslinked zwitterionic gel of claim 1, wherein the radical crosslinked zwitterionic is self-healing by reestablishing crosslinking.

15. The radical crosslinked zwitterionic gel of claim 1, wherein the radical crosslinked zwitterionic gel is cytocompatible.

16. The radical crosslinked zwitterionic gel of claim 1, the radical crosslinked zwitterionic gel has a storage modulus from about 500 Pa to about 2,000 Pa.

17. The radical crosslinked zwitterionic gel of claim 1, wherein the radical crosslinked zwitterionic gel has an elastic modulus from about 1,000 Pa to about 20,000 Pa.

18. The radical crosslinked zwitterionic gel of claim 1, wherein the radical crosslinked zwitterionic gel swells in the presence of an aqueous solution.

19. The radical crosslinked zwitterionic gel of claim 1, wherein the radical crosslinked zwitterionic gel releases the therapeutic agent over an extended period.

20. A method of preparing a radical crosslinked zwitterionic gel, the method comprising:
(a) contacting a zwitterionic monomer, a non-zwitterionic ionic monomer, an optional cross linking agent, an optional initiator, an optional rheology modifier, an optional antioxidant, and water forming a reaction mixture;
(b) optionally filtering the reaction mixture through a 0.22 μm pore filter to form a filtered reaction mixture;
(c) exposing the reaction mixture and/or filtered reaction mixture to light or other suitable radical generator thereby polymerizing the zwitterionic monomer and the non-zwitterionic ionic monomer to form a radical crosslinked zwitterionic gel;
(d) optionally washing the radical crosslinked zwitterionic gel to form a washed crosslinked zwitterionic gel; and
wherein the radical crosslinked zwitterionic gel and or washed crosslinked zwitterionic gel has an average pore size greater than about 50 mm and less than about 100 mm.

21. The method of claim 20, wherein the zwitterionic monomer selected from the group consisting of [2-(methacryloyloxy) ethyl]dimethyl-(3-sulfopropyl)ammonium hydroxide (SBMA), [(methacryloyloxy)ethyl] dimethylammonio]propionate (CBMA), 2-Methacryloyloxyethyl phosphorylcholine (MPC), and combinations thereof.

22. The method of claim 20, wherein the non-zwitterionic monomer is hydroxyethyl methacrylate (HEMA).

23. The method of claim 20, further comprising adding a crosslinking agent.

24. The method of claim 23, wherein the crosslinking agent is selected from a group consisting of glycerol dimethacrylate (GDMA), methylene bisacrylamide (MBA), polyethylene glycol dimethacrylate (PEGDMA), and a combination of two or more crosslinkers.

25. The method of claim 20, wherein the crosslinking agent is polyethylene glycol dimethacrylate (PEGDMA).

26. The method of claim 20, further comprising adding a radical initiator.

27. The method of claim 26, wherein the initiator is selected from a group consisting of sodium persulfate, ruthenium (II)-tris(2,2′-bipyridyl) dichloride, and a combination thereof

28. The method of claim 20, wherein the light is near UV light, visible light, or white light.

29. The method of claim 20, wherein the chemical free radical generator is ammonium persulfate (APS), N,N,N′N′-tetramethylethylene-1,2-diamine, an organic peroxide (TEMED), ascorbic acid, and combinations thereof.

30. The method of claim 20, wherein the antioxidant is resveratrol.

31. The method of claim 20, wherein the rheology modifier is a clay.

32. The method of claim 20, wherein the radical crosslinked zwitterionic gel is washed with a buffered aqueous saline solution.

33. The method of claim 32, wherein the buffered aqueous saline solution comprises phosphate buffered saline.

34. The method of claim 20, wherein the method is conducted at room temperature.

35. A method of treating a wound, the method comprises administering or applying the radical crosslinked zwitterionic gel of claim 1 to a wound of a subject in need thereof.

36. The method of claim 35, wherein the wound is a diabetic ulcer.
